(19) United States
(12) Patent Application Publication
(10) Pub. No.: US 2014/0352703 A1
(43) Pub. Date: Dec. 4, 2014

(54) APPARATUS AND METHODS FOR PREVENTING OCULAR DEHYDRATION IN ANESTHETIZED SMALL ANIMALS

(71) Applicant: THE CLEVELAND CLINIC FOUNDATION, Cleveland, OH (US)

(72) Inventor: Brent A. Bell, Cleveland, OH (US)

(73) Assignee: THE CLEVELAND CLINIC FOUNDATION, Cleveland, OH (US)

(21) Appl. No.: 14/279,390

(22) Filed: May 16, 2014

Related U.S. Application Data
(60) Provisional application No. 61/828,324, filed on May 29, 2013.

(51) Int. Cl. A61F 9/04

(52) U.S. Cl. CPC A61F 9/04
USPC 128/858

(57) ABSTRACT

One aspect of the present disclosure relates a protective eye cup that protects a small animal's eye from developing an ocular opacity in the refractive lens during a vision research experiment. The protective eye cup can include at least a dome and a shoulder. The dome can be shaped with an outer geometry configured to facilitate placement and removal of the protective eye cup. The dome can also be shaped with a hemispherical inner geometry with a height and a radius selected to cover the eye. The inner geometry can define a hollow reservoir configured to hold the eye and an associated hydrating fluid when positioned on the eye. The shoulder can provide a radial edge to the dome that increases a contact surface area of the portion of the dome in contact with the hydrating fluid. The protective eye cup can attach to a region external to the eye based on a surface tension of the hydrating fluid.

### RELATED APPLICATIONS

This application claims the benefit of U.S. Provisional Application No. 61/828,324, filed May 29, 2013, entitled “Apparatus and Methods for Preventing Cataract Formation in Rodents Undergoing Ocular Imaging and Vision Research Testing Procedures,” the entirety of which is hereby incorporated by reference for all purposes.

### GOVERNMENT FUNDING

This work was supported, at least in part, by grant number NIH R01EY014240 from the Department of Health and Human Services, National Institutes of Health. The United States government may have certain rights in this invention.

### TECHNICAL FIELD

The present disclosure relates generally to protecting the eyes of anesthetized small animals during in vivo intraocular imaging studies and, more specifically, to an apparatus and methods for protecting the eyes of anesthetized small animals during in vivo intraocular imaging studies to prevent dehydration of ocular tissue and to minimize the development of ocular opacities.

### BACKGROUND

An anesthetized small animal generally cannot exhibit a blink reflex, which can allow the tear film on the cornea to evaporate. As a result, during the course of an imaging study, the corneal surface of the small animal's eye can dehydrate within minutes. Without supplemental hydration, the eye can develop ocular opacities (e.g., anterior lens cataracts) that can compromise experiments, such as in vivo intraocular imaging studies.

While these ocular opacities are generally reversible, excessive exposure to ambient room air without the ability to blink can lead to damage (e.g., corneal ulceration) that can render the animal useless for follow up imaging studies. In certain circumstances, ocular opacities can negatively impact image quality to the point that the information collected is uninformative and/or unusable. For instance, ocular opacities can affect the transparency of the cornea and obscure visibility of at least a portion of the retina.

Different varieties of contact lenses have been designed to prevent the formation of cataracts during small animal intraocular imaging studies. Generally, the contact lenses can be constructed from a reusable, optical quality, plastic (e.g., PMMA) of a thickness on the millimeter level. The contact lenses can that fit directly onto the corneal surface of a small animal's eye. However, the contact lenses generally are rigid devices that do not conform perfectly to the corneal surface of the small animal's eye leaving one or more regions (e.g., peripheral regions) unprotected. The unprotected regions are exposed to ambient room air, which can lead to dehydration and the formation of ocular opacities.

### SUMMARY

The present disclosure relates generally to protecting the eyes of anesthetized small animals during in vivo intraocular imaging studies. More specifically, the present disclosure relates to an apparatus and methods for protecting the eyes of anesthetized small animals during in vivo intraocular imaging studies to prevent dehydration of ocular tissue and to minimize the development of ocular opacities.

In one aspect, the present disclosure can include a protective eye cup apparatus that protects an eye of a small animal from developing an ocular opacity in the refractive lens during a vision research experiment. The protective eye cup can include at least a dome and a shoulder. The dome can be shaped with an outer geometry configured to facilitate placement and removal of the eye cup. The dome can also be shaped with a hemispherical inner geometry with parameters (e.g., overall dome height and radius of the dome opening) selected to cover the eye. The inner geometry defines a hollow reservoir configured to contain the eye completely and an associated hydrating fluid when positioned over the eye. The shoulder can be configured to provide a radial edge to the dome that increases a contact surface area of the portion of the dome in contact with the hydrating fluid and extraocular surface of the eye. The protective eye cup apparatus can attach to the eye based on a surface tension of the hydrating fluid.

In another aspect, the present disclosure can include a method for protecting an eye of a small animal from developing an ocular opacity in the refractive lens during a vision research experiment. The method can include one or more steps that can include: anesthetizing the small animal; applying a hydrating fluid to the eye; and covering the hydrating fluid and the eye with a protective eye cup apparatus. The protective eye cup apparatus can include at least a dome and a shoulder. The dome can be shaped with an outer geometry configured to facilitate placement and removal of the eye cup. The dome can also be shaped with a hemispherical inner geometry with parameters (e.g., overall dome height and radius of the dome opening) selected based on the size of the eye. The inner geometry defines a hollow reservoir configured to contain the eye and an associated hydrating fluid when positioned over the eye. The shoulder can be configured to provide a radial edge to the dome that increases a contact surface area of the portion of the dome in contact with the hydrating fluid and the extraocular surface of the eye. The protective eye cup apparatus can attach to the eye based on a surface tension of the hydrating fluid.

In a further aspect, the present disclosure can include a system that protects an eye of a small animal from developing an ocular opacity in the refractive lens during a vision research experiment. The system can include a hydrating fluid that moistens the eye; and a protective eye cup apparatus configured to hold the hydrating fluid and the eye when positioned over the eye. The protective eye cup apparatus attaches to the eye based on a surface tension of the hydrating fluid and covers the entire eye of the small animal.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a cross-sectional diagram showing an eye cup apparatus that can protect an eye of an anesthetized small animal during an in vivo intraocular imaging study to prevent dehydration of ocular tissue and to minimize the development of ocular opacities in accordance with an aspect of the present disclosure;

FIGS. 2-5 are cross-sectional diagrams showing alternative designs of the eye cup apparatus of FIG. 1;

FIG. 6 is a process flow diagram illustrating a method for preventing dehydration of ocular tissue and minimizing the development of ocular opacities in accordance with another aspect of the present disclosure;

FIG. 7 is a process flow diagram illustrating a method for the pre-imaging preparation of FIG. 6;

FIG. 8 is a process flow diagram illustrating a method for the imaging session of FIG. 6;

FIG. 9 is a process flow diagram illustrating a method for the post-imaging care of FIG. 6;

FIG. 10 depicts an example of the eye cup apparatus of FIG. 2 designed to fit a mouse eye;

FIG. 11 depicts an example of the eye cup apparatus of FIG. 2 designed to fit a rat eye;

FIG. 12 is a representative depiction of the utility of the eye cup apparatus against opacity formation during an experiment with an anesthetized wild type (C57BL/6J) mouse;

FIG. 13 is a plot of media (anterior lens) opacity changes as a function of time for protected and unprotected eyes in anesthetized wild type mice (n=4); and

FIG. 14 includes representative digital photographs (oblique view taken from the nasal direction) of the eyes of two mice immediately after undergoing the experiment shown in FIG. 12.

### DETAILED DESCRIPTION

### I. Definitions

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups. As used herein, the term “and/or” can include any and all combinations of one or more of the associated listed items. Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ocular opacity” can refer to a condition in which light cannot completely pass through a portion of the eye (e.g., the cornea and/or the lens). In some instances, the ocular opacity can be caused by ocular dehydration and/or desiccation due at least in part to the lack of a blink reflex due to anesthesia (e.g., allowing the tear film in the cornea to evaporate), which exposes the portion of the eye to room air. In some instances, the ocular opacity can be reversible. In other instances, the ocular opacity can be irreversible. An example of a type of ocular opacity is a cataract (a clouding of the lens). The terms “ocular opacity” and “lens opacity” can be used interchangeably herein. Additionally, when used herein, dehydration can also include desiccation.

As used herein, the term “intraocular imaging study” can refer to a non-invasive technique to document the progression of ocular changes in small animals in vivo. Examples of imaging modalities that can be used for an intraocular imaging study include: optical coherence tomography (OCT) and scanning laser ophthalmoscopy (SLO). In some instances, the term intraocular imaging study can also include light dosimetry studies.

As used herein, the term “in vivo” can refer to an experiment using a whole, living subject.

As used herein, the term “subject” can refer to any research small animal undergoing an in vivo intraocular imaging study.

As used herein, the term “small animal” can refer to any type of research animal that can be used for an in vivo intraocular imaging study. Examples of small animals can include: rodents, salamanders, rabbits, dogs, cats, etc.

As used herein, the term “rodent” can refer to a mammal of the order rodentia that are characterized by a single pair of continuously growing incisors in each of the upper and lower jaws. Examples of rodents include rats and mice.

As used herein, the term “eye cup” can refer to an apparatus that can cover the eyes of the small animal to prevent dehydration of ocular tissue and minimize the formation of ocular opacities during an intraocular imaging study. The terms “eye cup” and “eye shield” can be used interchangeably herein.

As used herein, the term “hydrating fluid” can refer to an aqueous, viscous, or semi-viscous ophthalmic preparation (e.g., fluid, drops, and/or ointment). The hydrating fluid can act as a binding agent to facilitate the attachment of the eye cup to the eye of a small animal. Example formulations that can be used as the hydrating fluid can include one or more of: Phosphate Buffered Saline (PBS), Balanced Salt Solutions (BSS), polyethylene glycol (PEG), propylene glycol, carboxymethylcellulose sodium, hypromethylcellulose, petroleum, mineral oil, Polymyxin B, or Bacitracin Zinc.

### II. Overview

The present disclosure relates generally to protecting the eyes of anesthetized small animals during in vivo intraocular imaging studies. More specifically, the present disclosure relates to an apparatus and methods for protecting the eyes of anesthetized small animals from dehydration and minimizing the development of ocular opacities during in vivo intraocular imaging studies.

Non-invasive in vivo small animal optical imaging techniques can be used in eye research to document disease-related changes in small animal eyes. During ocular imaging studies, generally, the small animals are put under general anesthesia. Anesthetized small animals often lose their blink reflex. As a consequence, the small animal cannot maintain a tear film over its eyes, and drying occurs so that the cornea becomes dehydrated, which can be followed by structural damage to the cornea (e.g., resulting in ocular opacities) without supplemental hydration. These changes can compromise image quality, especially for imaging studies involving repeated use of the same animal over several weeks or months. A protective eye cup apparatus, sized and dimensioned to cover the eye of the small animal, can minimize development of ocular dehydration.

The protective eye cup apparatus, in connection with a hydrating fluid, can be placed over the entirety of the eye (attaching to the exterior of the eye), when the anesthesia immobilizes the animal. The eye cup apparatus can be removed for the brief periods required to conduct the imaging study and then reapplied before the fellow eye is examined. As a result, the corneal surface of each eye can be exposed only for the time period required for the imaging study. When the eye cup apparatus and associated methods are used consistently, high quality images can be obtained repeatedly from individual animals.

### III. Apparatus

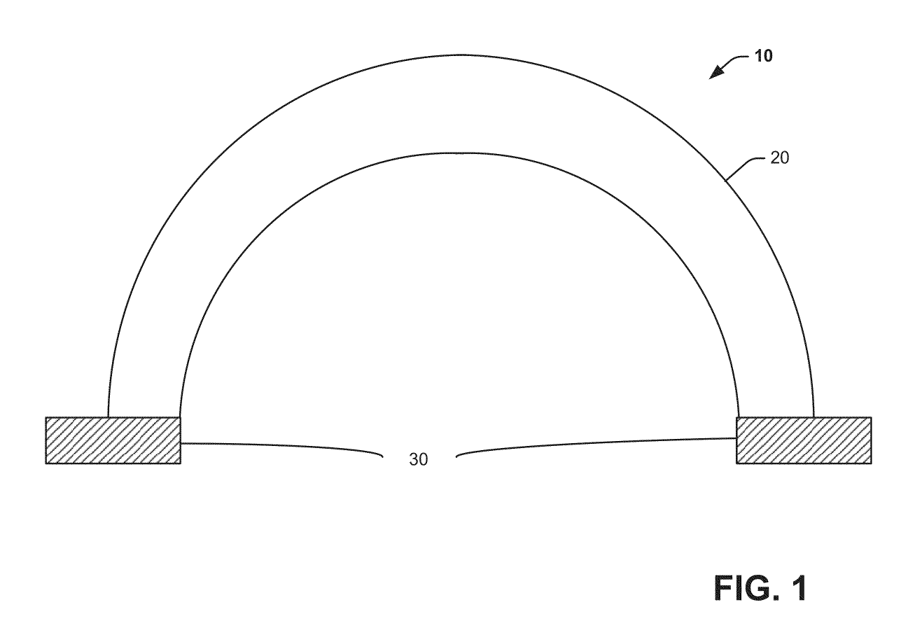

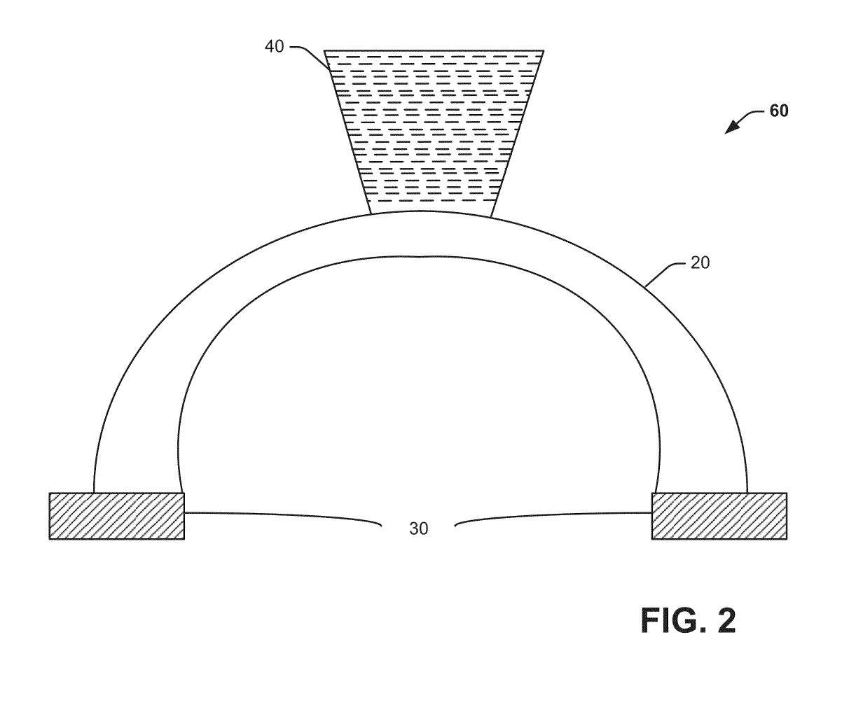

One aspect of the present disclosure can include an eye cup apparatus that can protect an eye of an anesthetized small animal from dehydration during an in vivo intraocular imaging study. The eye cup apparatus can provide a barrier that can minimize the eye's exposure to room air (e.g., preventing evaporation of fluid on the cornea) to prevent dehydration and to minimize the development of ocular opacities. An example configuration of the eye cup apparatus 10 is shown in FIG. 1. Alternate configurations of the eye cup apparatus 60, 70, 80, and 90 are shown in FIGS. 2, 3, 4, and 5, respectively.

FIGS. 1-5 are schematic cross-sectional illustrations of different configurations of the eye cup apparatus 10, 60, 70, 80, and 90, with like reference numbers designating like or similar parts throughout. The FIGS. 1-5 are not intended to be drawn to scale. For example, purely for the sake of greater clarity, wall thickness and spacing are not dimensioned as they actually exist in the assembled eye cup.

As shown in FIG. 1, one aspect of the present disclosure can include an eye cup apparatus 10 configured to protect an eye of an anesthetized small animal during an in vivo intraocular imaging study from dehydration. The eye cup apparatus can include a dome 20 and a shoulder 30. In some instances the eye cup apparatus 10 can be preloaded or used in combination with a hydrating fluid to can keep the eye hydrated. The dome 20 can be configured to cover the eye and the associated hydrating fluid. The shoulder 30 can be configured to facilitate attachment of the eye cup apparatus 10 to the exterior of the eye (e.g., a region surrounding the eye).

In some instances, the eye cup apparatus 10 can be sized and dimensioned to fit over the exterior of the eye. Unlike a contact lens, the eye cup apparatus 10 is not in direct contact with the cornea. In some instances, the eye cup apparatus 10 can fit over the eyelid. Although not wishing to be bound by theory, it is believed that a mild vacuum effect that occurs when pressing the eye cup apparatus 10 onto the region surrounding the eye, combined with the surface tension properties of the hydrating fluid, can serve to hold the eye cup apparatus 10 in place over the eye for an extended period of time without the risk of falling off once it is placed over the eye to cover the eye.

The eye cup apparatus 10 can be placed over the eye of the anesthetized animal before an imaging procedure to protect against dehydration of the ocular tissue and the development of ocular opacities. Each individual eye cup apparatus can be affixed to a single eye of the small animal. A different and unique instance of the eye cup apparatus can be affixed to the fellow eye of the small animal. In some instances, however, two different and unique eye cup apparatuses can be included in a common device that covers each of the eyes with one of the eye cups. The eye cup apparatus 10 can be removed during the imaging procedure and replaced after the imaging procedure. In some instances, the eye cup apparatus 10 can be left on the eye until the animal has recovered from the effects of the general anesthesia and can knock the eye cup apparatus 10 off its eye.

As shown in FIG. 1, the eye cup apparatus 10 can include a dome 20 configured to cover the eye and a shoulder 30 configured to attach to the exterior of the eye. The dome 20 can include a hollow, potential space that can be sized and dimensioned to cover the eye of the small animal. The hollow, potential space can serve as a hollow reservoir that can hold the eye and the associated hydrating fluid. The hollow, potential space can be sized and dimensioned to hold the eye and the associated hydrating fluid.

The dome 20 can include an outer geometry and an inner geometry. In some instances, such as illustrated in FIG. 1, the outer geometry and the inner geometry can be generally the same shape with different sizes. However, in other instances, the outer geometry and the inner geometry can have different shapes.

The outer geometry can be configured to facilitate placement of the eye cup apparatus 10 on the exterior of the eye and removal of the eye cup from the eye. The outer geometry of the eye cup apparatus 10, as illustrated, can be a hemispherical shape. However, the outer geometry can also be an ellipsoidal shape, a hyperbolic shape, a square shape, a rectangular shape, and/or a polygonal shape. In some instances, the outer geometry can be tapered to facilitate the placement and removal of the eye cup apparatus 10 from the eye (e.g., by human fingers or mechanical forceps).

The inner geometry can define the hollow reservoir configured to hold the eye of the small animal and the associated hydrating fluid. In some instances, such as illustrated in FIG. 1, the inner geometry can be of a hemispherical shape sized and dimensioned to hold the eye. However, in other instances, the inner geometry can be generally hemispherical with a portion added or altered from hemispherical.

The inner geometry can be defined by parameters, including an overall dome height and a radius of the dome opening. These parameters can be selected so that the dome 20 can cover the eye. In some instances, the dome height and radius of the dome opening can be selected based on the type of small animal (e.g., different species of small animal can require domes of different sizes to cover the eye completely). For example, when the dome 20 is used with mice, the dome height can be equal to the radius of the dome opening. In another example, when the dome 20 is used with rats, the dome height can be less than the radius of the dome opening, yielding a shallower dome with a lesser volume.

In some instances, the dome 20 can be constructed from one or more materials that are optically opaque or dense to shield the eye from ambient or bright light. For example, the materials can include one or more plastic materials with different gas permeability. In another example, the materials can include one or more materials with special drug eluding properties. The hydrating fluid used with the opaque dome can be pigmented black.

In other instances, the dome 20 can be constructed from one or more materials that can be colored for better visibility and/or to filter the light being transmitted through the eye during the imaging experiment. For example, at least a portion of the dome can have glow in the dark properties, which can provide a better visibility of the dome 20 in a dark room.

In further instances, at least a portion of the dome 20 can be optically transparent (e.g., transparent to ultraviolet light, visible light, and/or near-infrared light). The optically transparent portion can allow the dome 20 to remain covering the eye during the imaging study. The optically transparent portion can be constructed from one or more transparent materials, such as: polystyrene, poly-methyl methacrylate (PMMA), polycarbonate, or glass. The hydrating fluid used with the clear dome can be clear.

In some instances, a combination of eye cup apparatuses with light opaque domes and/or transparent domes can be used during an imaging study. For example, one eye can be exposed a particular light dose while the fellow eye can be shielded and used as a control.

The shoulder 30 of the eye cup apparatus 10 can facilitate attachment of the protective eye cup to the exterior of the eye. The shoulder 30 can extend radially from the dome 20. The shoulder 30 can have a round, square, rectangular or polygonal shape extending from the end of the dome. The geometrical configuration and/or size of the shoulder 30 can be selected according to the type and/or size of the small animal. In some instances the shoulder 30 can be virtually flat (vertically) compared to the dome 20)

The shoulder 30 can be configured to provide a smooth radial edge to the dome. The shoulder 30 can facilitate attachment of the eye cup apparatus 10 to the exterior of the eye (e.g., to a region surrounding the eye) based on a surface tension of the hydrating fluid that can be used in connection with the eye cup apparatus 10. In some instances, the radial edge provided by the shoulder 30 can be atraumatic and/or rounded to prevent damage when the eye cup apparatus 10 is placed over the eye. The radial edge can increase a contact surface area of the portion of the dome in contact with the area immediately surrounding the eye to enhance the adhesion properties of the eye cup apparatus 10. The shoulder can extend radially from the edge of the dome to increase the surface tension adhesion for better attachment to the exterior of the eye.

As shown in FIG. 2, an eye cup apparatus 60 can include the dome 20 and the shoulder 30, as described above. The eye cup apparatus 60 can also include a post 40 or stem that can facilitate grasping the eye cup apparatus. In some instances, the post 40 can be positioned directly over the pole or top of the dome. In other instances, the post 40 can be positioned to the side of the dome (e.g., not directly over the pole) so that imaging can be performed through the central axis of the dome 20. The post 40 can facilitate handling and manipulation of the eye cup apparatus 10 by human fingers and/or by mechanical means (e.g., forceps). For example, the post 40 can serve as a grasping point to pick up and/or manipulate the eye cup apparatus (e.g., for easy placement and removal of the eye cup apparatus 10 during ocular imaging experiments). To increase the grasping friction, in some instances, the post can be at least partially tapered.

In some instances, the post 40 can be constructed from a material that is optically transparent (e.g., polystyrene, poly-methyl methacrylate (PMMA), polycarbonate, or glass). In other instances, the post 40 can be constructed from one or more materials that can be colored for better visibility (e.g., the material can possess glow in the dark properties).

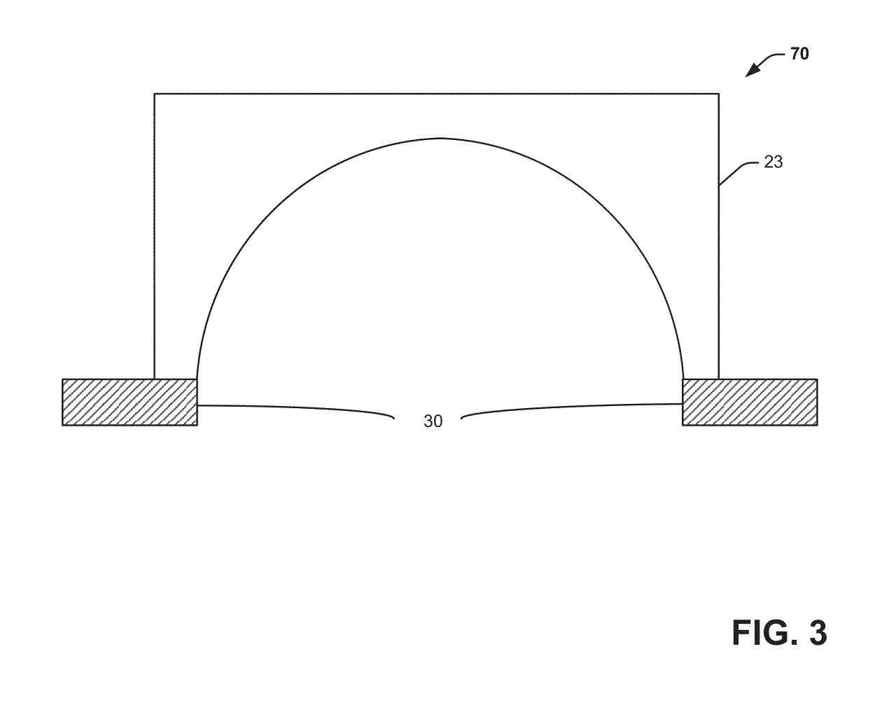

FIG. 3 depicts an example of an eye cup apparatus 70 that includes a dome 23 of a having a polygonal outer geometry, while maintaining the hemispherical inner geometry, and the shoulder 30. As illustrated in FIG. 3, the outer geometry of the dome 23 can have a box-like outer geometry structure. The box-like outer geometry can be suited for grasping and manipulating the eye cup apparatus 70. In other instances, the outer geometry can be a different polygonal shape that can facilitate grasping and manipulating the eye cup apparatus 70. For example, symmetrical polygons (e.g., a hexagon, an octagon, etc.) can facilitate easy grasping with small mechanical forceps. In some instances, the outer geometry can be tapered to prevent forceps from slipping during grasping and manipulation of the eye cup apparatus 70.

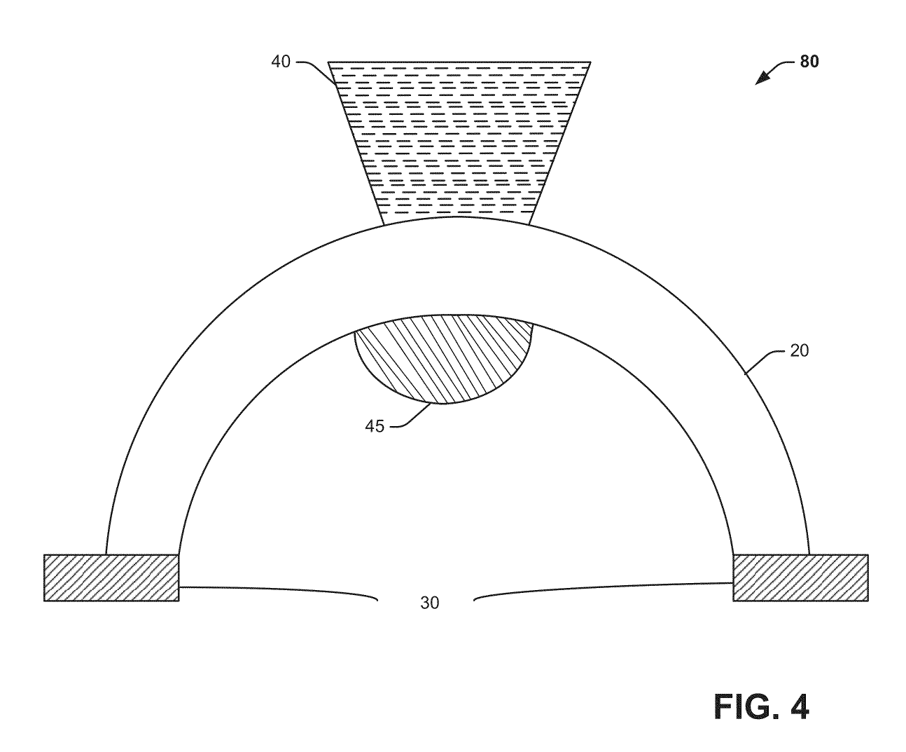

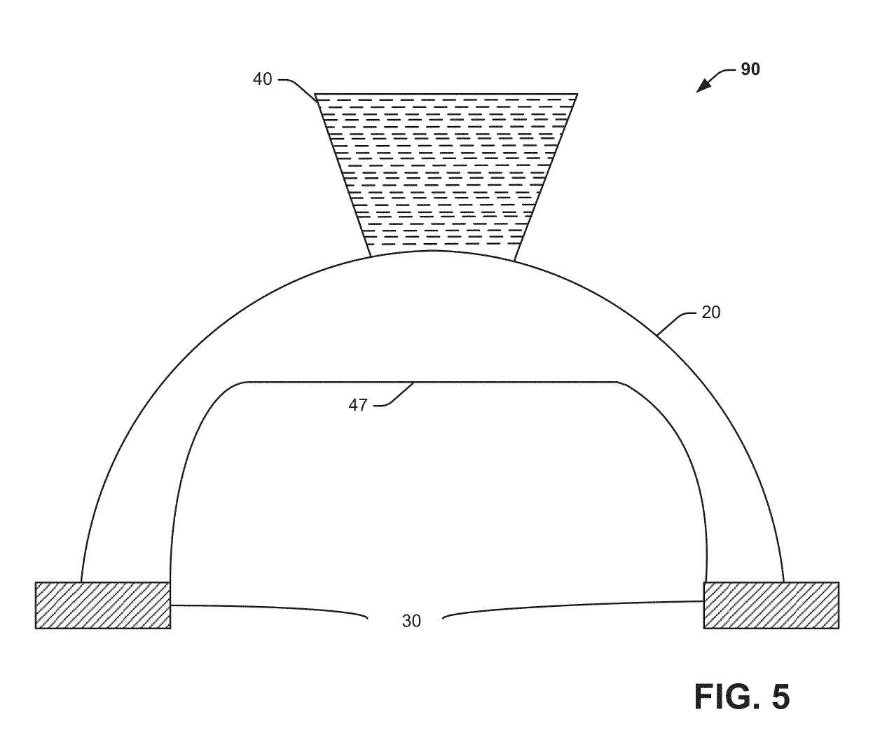

FIGS. 4 and 5 each depict examples of the eye cup apparatus 80 and 90 that include the dome 20 with modified inner geometries 45, 47 and the shoulder 30. Both examples of the eye cup apparatus 80, 90 are intended to counteract the proptosis or luxating effect that can be observed when anesthesia or mydriatic drugs (e.g., muscle relaxing agents that are adrenergic receptor agonists can cause muscles that normally work to hold the eye in the socket to relax and cause the eye to proptose) are used on the small animal for the purpose of inducing general anesthesia and pupil dilation, respectively. The modified inner geometries 45, 47 are each generally hemispherical shaped within a non-hemispherical portion. The non-hemispherical portions can apply a mild force to the exterior and/or corneal surface of the eye to counteract the proptosis. The modified inner geometry 45 of FIG. 4 can include a rounded pip that can apply pressure to the corneal surface. The modified inner geometry 47 of FIG. 5 can include a flat surface that can achieve the same goal as the pip of FIG. 4.

### IV. Methods

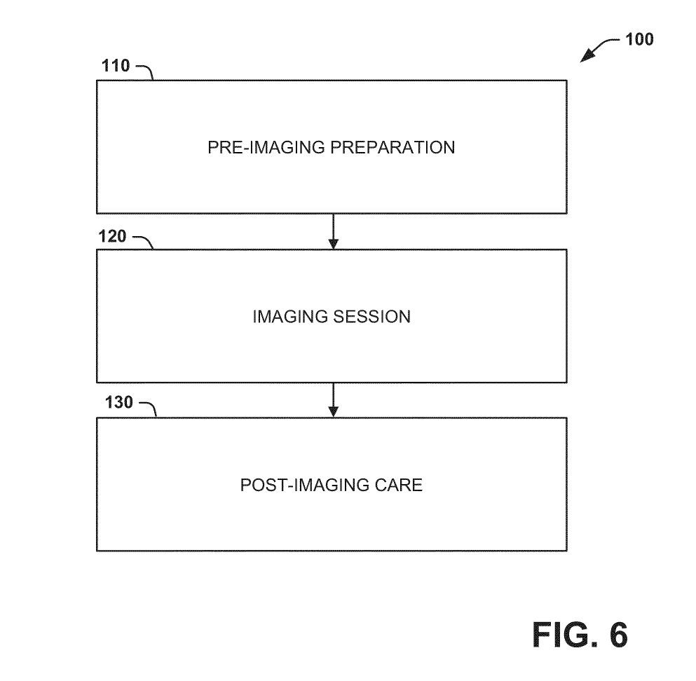

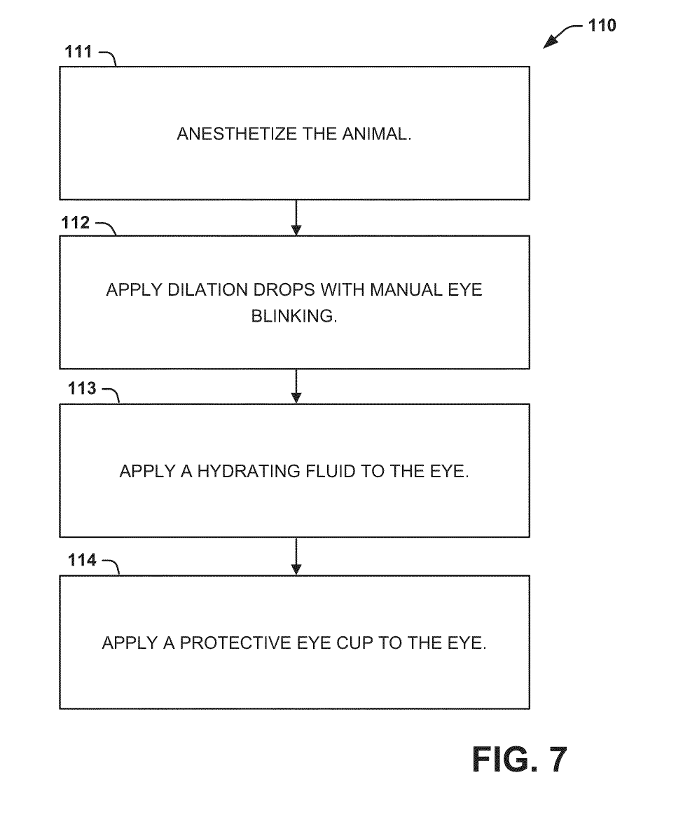

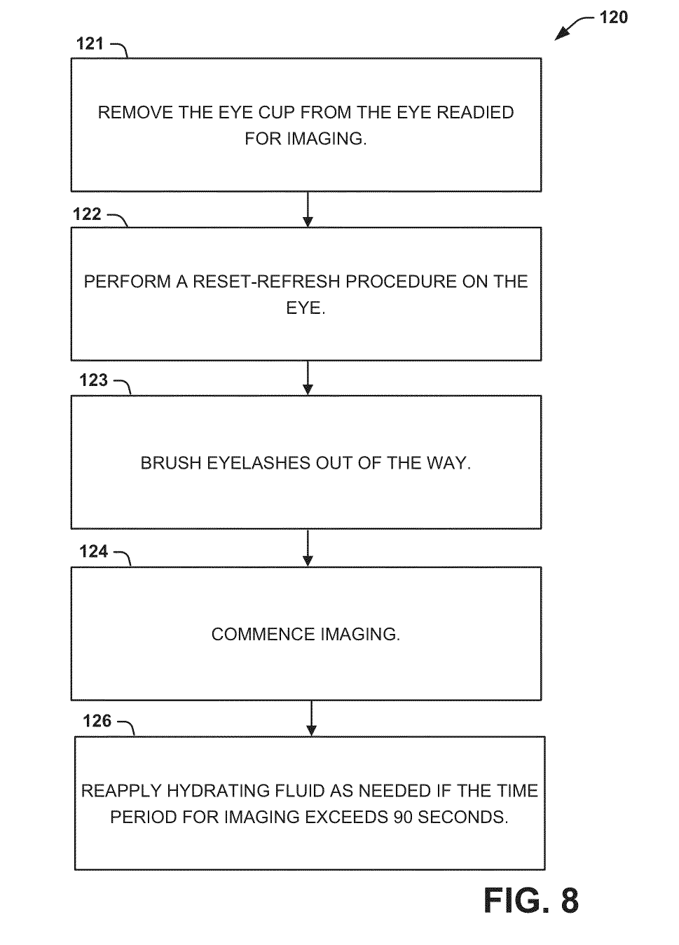

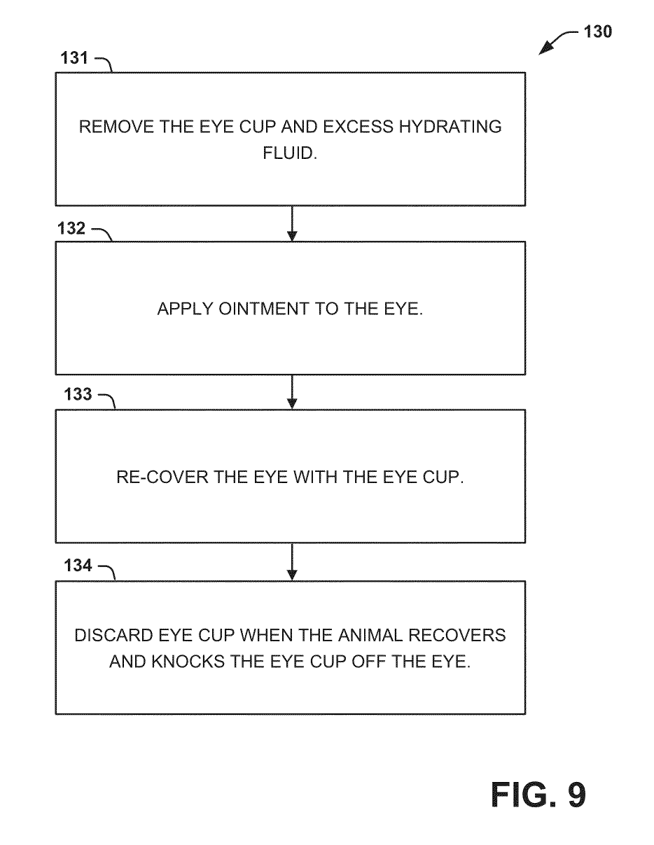

A second aspect of the present disclosure can include methods that can protect the eyes of anesthetized small animals during in vivo intraocular imaging studies to prevent dehydration of the ocular tissue and to minimize the development of ocular opacities. An example of a method 100 that can prevent dehydration of ocular tissue and to minimize the development of ocular opacities is shown in FIG. 6. Examples of the steps of method 100 are further delineated in method 110 of FIG. 7, method 120 of FIG. 8, and method 130 of FIG. 9. The steps of method 110 can accomplish the pre-imaging preparation of FIG. 6. The steps of method 120 can accomplish the imaging session of FIG. 6. The steps of the method 130 can accomplish the post-imaging care of FIG. 6.

The methods 100, 110, 120, and 130 are illustrated as process flow diagrams with flowchart illustrations. For purposes of simplicity of explanation, the methods 100, 110, 120, and 130 are shown and described as executing serially, it is to be understood and appreciated that the present disclosure is not limited by the illustrated order, as some aspects could occur in different orders and/or concurrently with other aspects shown and described herein. Moreover, not all illustrated aspects may be required to implement methods 100, 110, 120, and 130.

Referring to FIG. 6, an aspect of the present disclosure can include a method 100 preventing dehydration of ocular tissue of a small animal. By preventing the dehydration of the ocular tissue, the method 100 can minimize the development of ocular opacities during an imaging session.

An imaging session can be divided into three distinct phases that each incorporates procedures that are important for optimal maintenance of the eye, quality of the image and recovery of animal for additional imaging at a later time. The first phase involves pre-imaging preparation (method 110). The second phase involves conducting the imaging session (method 120). The third phase involves post-imaging care (method 130).

FIG. 7 shows an example of the pre-imaging preparation process (method 110). The steps of the pre-imaging preparation can occur in a different order than that illustrated. Additionally, although the pre-image preparation is described with respect to a single eye, the pre-imaging preparation can be applied to both eyes.

At step 111, the animal can be anesthetized. To anesthetize the animal, the animal weight can be obtained so that the correct anesthesia dose can be prepared and administrated. In some instances, after administration of the anesthetic, the animal can be placed into a humidified, bottom-warmed, holding chamber to wait for anesthesia to take effect (e.g., the animal can become inactive and immobilized).

At step 112, dilation drops can be applied to the eye. A manual eye blinking process can be applied to ensure even distribution of the dilation drops over the cornea to promote uniform absorption. In some instances, the dilation drops can be applied to the eye after the anesthesia takes effect. In other instances, the dilation drops can be applied prior to the anesthetization of the animal.

At step 113, a hydrating fluid can be applied to the eye. At step 114, the protective eye cup can be applied to the eye to cover the entire eye. In some instances, after covering the eye, the animal can be returned to the chamber/warming plate until the pupil is dilated.

FIG. 8 shows an example of the imaging session (method 120). The steps of the imaging session can occur in a different order than that illustrated. The imaging session can begin when the animal is anesthetized and eye cups cover both eyes.

The animal can be transferred to an appropriate imaging holder. The eyes can be imaged one at a time, with the other remaining covered by the eye cup. The eye that will be imaged can be prepared for imaging by grossly aligning the eye in front of the imaging instrument.

At step 121, the eye cup can be removed from the eye readied for imaging. At step 122, a reset-refresh procedure can be performed on the eye. For example, the reset-refresh procedure can be undertaken if the eye cup has been left in place for an extended period of time. The reset-refresh procedure can include reapplying the hydrating fluid to the eye. Mechanical blinking can be applied to help remove the excess fluid. In some instances, excess fluid can be removed using cotton-tipped applicator.

At step 123, the eyelashes (e.g., upper eyelashes that are not visible to the naked eye) can be brushed out of the way (e.g., so the eyelashes are removed from the imaging path). An example procedure for brushing the eyelashes out of the way includes an upward sweeping motion from nasal to temporal, while also introducing some ventral to dorsal action. At step 124, the imaging procedure can be commenced. At step 126, if the imaging time period exceeds 90 seconds, the hydrating drops can be reapplied to moisten the cornea and, if necessary, a reset-refresh procedure can be applied again. Once the imaging procedure is completed on the first eye, the hydrating fluid and the eye cup can be reapplied to the eye. The imaging 120 session procedure can be repeated for imaging the other eye.

FIG. 9 shows an example of the post-imaging care (method 130). The steps of the post-imaging care can occur in a different order than that illustrated. Additionally, although the post-imaging care is described with respect to a single eye, the post-imaging care can be applied to both eyes after the imaging session to protect the animal against the lingering effects of general anesthesia.

At step 131, the eye cup (and excess hydrating fluid) can be removed from the eye. At step 132, an ointment can be applied to the eye. In some instances, the ointment can be a petroleum-based ophthalmic ointment. At step 133, the eye can be re-covered with the eye cup. The eye cup can help spread the ointment over the ocular surface.

The animal can be placed in a recovery chamber (e.g., a bottom-warmed, oxygenated chamber). As the animal recovers, it can displace the eye cup. The displacement of the eye cup can indicate the time when the animal has regained its blink response and is fully recovered. At step 134, the eye cup can be discarded when the animal recovers and knocks the eye cup off the eye.

### V. Examples

The following example is presented for the purpose of illustration only and is not intended to limit the scope of the appended claims.

### Example 1

This example demonstrates that protective eye cups of the apparatus described above (hereinafter referred to as “eye shields”), when used in conjunction with hydrating drops, can reduce a rodent's susceptibility to media opacity formation, as well as also potentially stabilize and/or slightly reduce existing opacities if they have already occurred.

### Methods

An imaging session can be divided into three distinct phases: (1) Pre-imaging Preparation, (2) Imaging, and (3) Post-imaging Care. Each phase incorporates key procedures that are important for optimal maintenance of the eye, quality of the image and recovery of the animal subject for additional imaging at a later time. The eye shields serve an integral and important part within each one of these phases of care.

### Pre-Imaging Preparation

Obtain the animal weight so that the correct anesthesia dose can be prepared and administrated. Place the mouse into a humidified, bottom-warmed, holding chamber and wait for anesthesia to take effect (˜5 minutes). Once inactive and immobilized, immediately remove animal from the chamber, apply mydriasis treatment to eyes (Note: dilation drops can also be applied prior to anesthesia induction as an alternative). Perform manual eyelid blinking to evenly distribute the dilation drops over the cornea and promote uniform absorption (Note: a few blinks are adequate). Trim vibrissae and apply one drop of semi-viscous artificial tears to each eye. Cover both eyes with protective eye shields and return animal to the chamber/warming plate and wait (˜5 minutes min) until pupils are dilated.

### Imaging Session

With eye shields covering both eyes, transfer the animal to the appropriate holder for imaging. Prepare the first eye for imaging by grossly aligning near the front of objective lens or beam path of the imaging instrument. Remove the cup from the eye to be imaged along with any residual hydration fluid using a cotton-tipped applicator and commence imaging. While imaging, reapply hydration drops as needed or if time period exceeds 90 seconds (Note: alternating between BSS and artificial tears seems to work well, i.e. if one type of hydrating fluid does not seem to improve the image, try the other one) and remove excess fluid using cotton-tip applicator. Once imaging is completed on the first eye, reapply viscous drops and the eye shield. Rotate animal for imaging the fellow eye and repeat the procedure on the fellow eye.

Occasionally, one may need to “refresh and reset” the eye if a shield has been left in place for an extended period of time. This can be accomplished best by applying a drop of saline and then mechanically blinking the lids a few times while simultaneously wicking away the excess fluid. After this procedure, the same process can be repeated with artificial tears. After smoothing the tear film by blinking it is helpful to finish by brushing away the upper eyelashes (which are not visible to the naked eye) with an upward sweeping motion from nasal to temporal simultaneously while introducing some ventral to dorsal action as well. This technique will ensure that they are removed from the optical imaging path.

### Post-Imaging Care

After imaging, the animal must be protected against the lingering effects of general anesthesia. Remove eye shields and excess fluid from both eyes and apply a petroleum-based ophthalmic ointment to the eyes. Cover with protective eye shields to smooth out ointment over the ocular surface. Place animal in a bottom warmed (30-33° C.), oxygenated (>21-40%) translucent chamber for recovery. As mice recover they will displace the eye shields which will indicate the time their blink response has returned and they are now ready to be returned to caging in the vivarium.

### Eye Shield Design, Prototyping and General Description

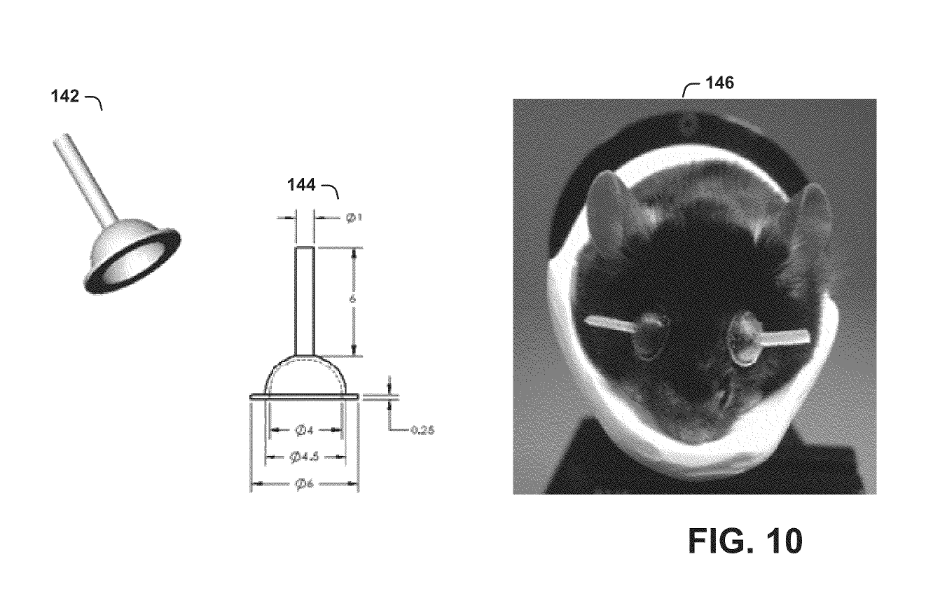

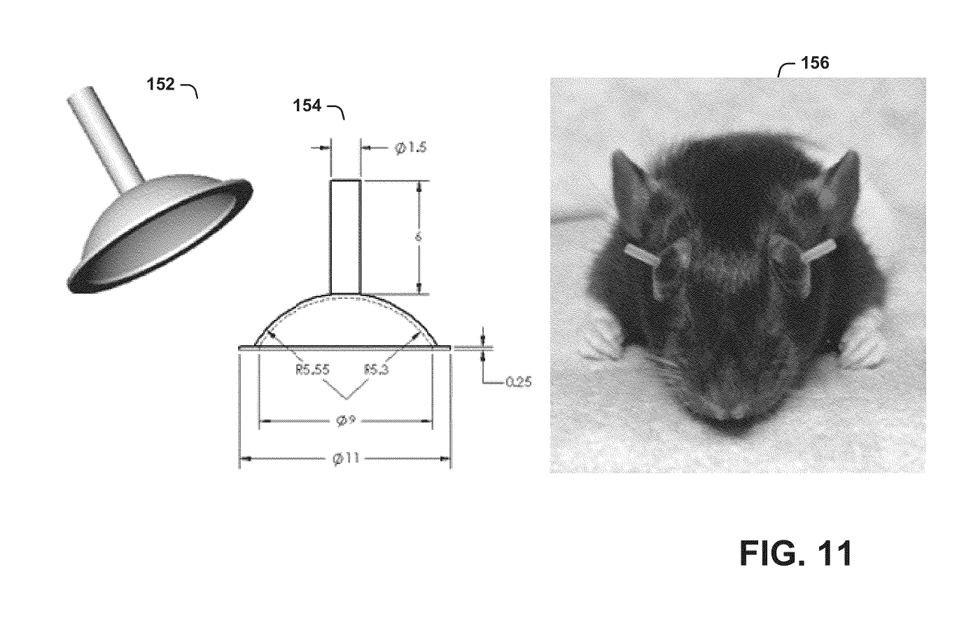

Hemispherical eye shields were designed and constructed using both measured and published eye dimensions for mice and rats. The dimensions of the hemispherical eye shield that were best suited for mice (presented in FIG. 10) and for rats (presented in FIG. 11). Two-dimensional CAD drawings were supplied to the Engineering Core Services at the Cleveland Clinic Lerner Research Institute. These drawings were converted into three-dimensional solid models using Solidworks 3D CAD (Dassault Systemes SolidWorks Corporation Waltham, Mass., USA). Rapid 3D prototypes were made with an Objet Eden260V printer using Fullcure720 resin (Stratasys, Eden Prairie, Minn., USA). In present form, the shields are reused over multiple imaging sessions until they are no longer functional (e.g., when the handling post breaks). Prototype shields on have been used with imaging of over 1700 animals (the vast majority of these animals were reimaged at later time points at least once, but up to a dozen times over a one year period after initial baseline imaging was performed) without observing any indication of adverse effects (e.g., toxicity) to the cornea from using the shields in this substantial cohort of animals.

The primary features of the eye shield include a hemispherical hollow dome (e.g., dome 20), a terminal shoulder (e.g., shoulder 30), and a grasping post (e.g., post 40) positioned directly at the apex of the hemispherical dome. The shield is designed to avoid direct contact with the cornea. For mice, the dome has a depth that is one-half the diameter (the radius) of the opening. For rats, the dome has a depth that is less than one-half the diameter of the opening. The shoulder provides a smooth, flared edge that avoids damage to the cornea. In addition, a flat surface on the shoulder increases the surface area and adhesion properties. The grasping post serves as an easy grasping point for manipulation either with forceps or by fingertips. The eye shield is constructed from a lightweight plastic and adheres to the eye by surface tension when used in conjunction with a single drop (15-30 μl) of ophthalmic fluid or ointments. The hollow cavity, which is designed to be larger than the eye, covers the eye and serves as a reservoir for excess hydration fluid. The hydration fluid can be a semi-viscous or “tacky” fluid (e.g., Systane® lubricating drops) and/or a viscous, petroleum/mineral oil based fluid (e.g., Refresh® or Lacri-lube® antibiotic ointments). Application of fluid, combined with positioning of the shield over the eye, creates a mild vacuum allowing the device to remain in position for extended periods without dislocation.

All experimental procedures and protocols involving animals were reviewed and approved by the Cleveland Clinic Institutional Animal Care and Use Committee. An experiment was performed on C57BL/6J mice (n=4) to demonstrate the utility of the eye shield. As soon as the mouse became immobile following anesthesia induction (65 mg/kg Sodium Pentobarbital), mydriasis treatment (1.5 μl drop/eye of 0.5% Mydrin®-P Tropicamide/Phenylephrine, Santen Pharmaceutical Co., Ltd, Japan) was administered to both eyes followed by the application of a single drop of Systane® Ultra (Alcon Laboratories, Ft. Worth, Tex.) in conjunction with prototype eye shields. Eye shields were kept in place for approximately 2 minutes while the mouse was transferred to an AIM-RAS (Animal Imaging Mount-Rodent Alignment System) mouse holder (Bioptigen, Inc., Durham, N.C., USA). A Spectral-Domain Optical Coherence Tomography System (Model 840HR SDOIS, Bioptigen, Inc., Durham, N.C., USA) was used to collect image volumes from the anterior segment of the eye, which included the cornea, iris and lens. Each OCT volume was 250 A-scans/B-scan by 250 B-scans/volume with imaging dimensions of 5 mm (azimuth)×5 mm (elevation)×2.3 mm (depth).

### Results

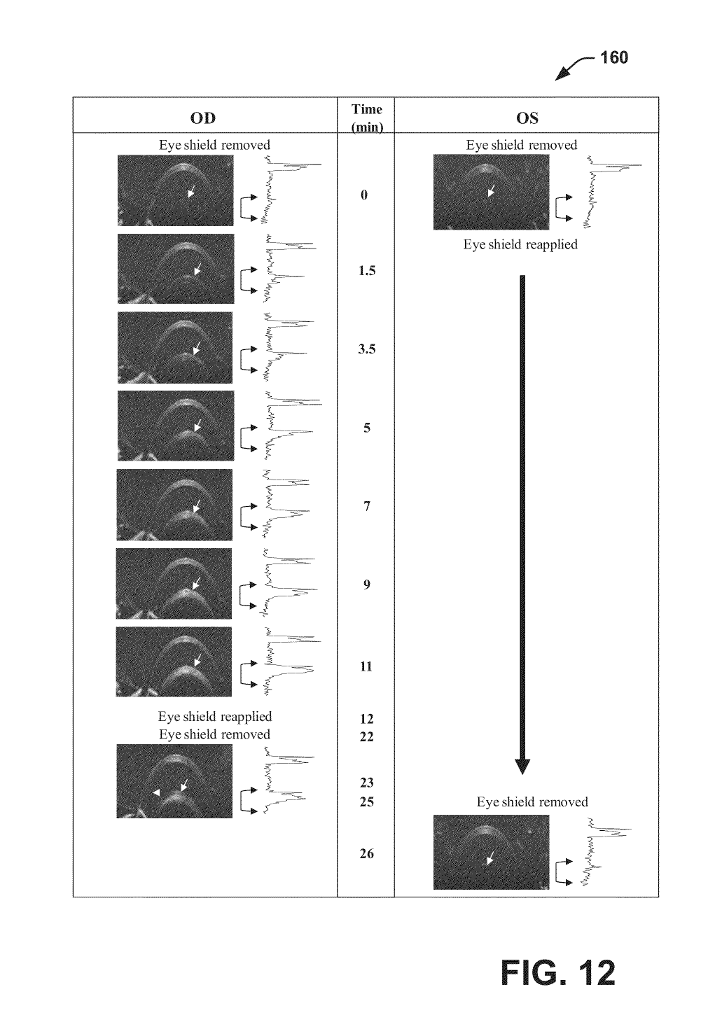

FIG. 12 shows representative OCT B-scan images from the experiment. Adjacent to each B-scan is an in-depth intensity profile showing back-scattered/reflected signal collected from the cornea, iris and lens. The intensity profiles were obtained from each B-scan using ImageJ (Rasband). Each profile is the average of 25 adjacent A-scans through the central cornea. Qualitative imaging data was converted into intensity profiles allowing the quantitative assessment of the magnitude of scatter as a function of depth. Scattering magnitude from the anterior lens region was determined using an area under the curve algorithm in Graphpad Prism v6.0a (GraphPad Software, Inc., La Jolla, Calif., USA).

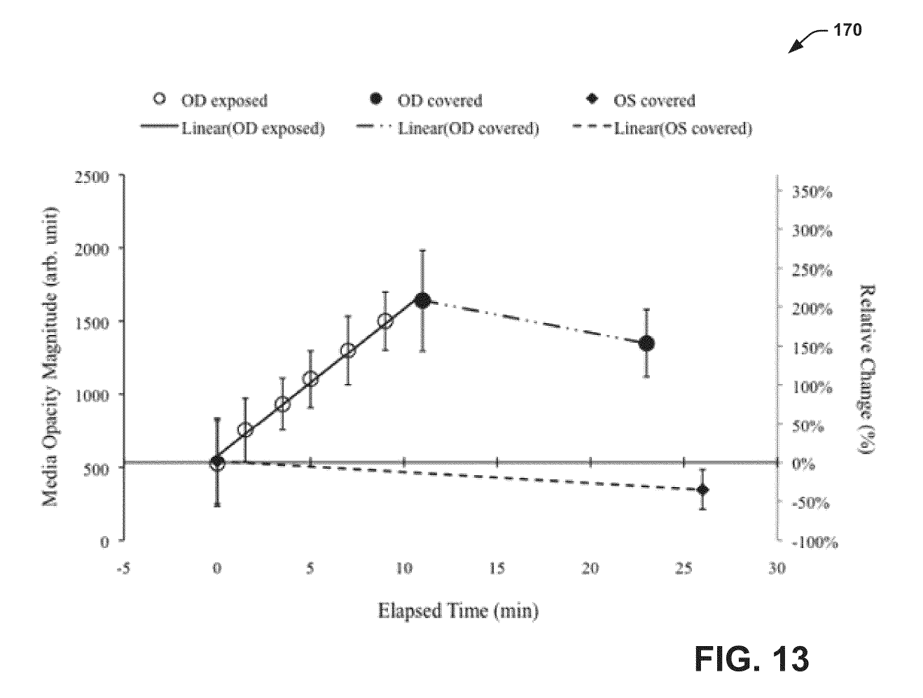

FIG. 12 illustrates the rapid development of anterior segment opacities in an unprotected versus eye shield protected eye. At baseline (t=0) the eye shield covering the left eye was removed and any residual fluid was removed from the cornea using a cotton-tipped applicator. The lids were mechanically blinked several times to restore a natural tear film on the cornea. An OCT B-scan was collected and the eye shield was immediately reapplied. This procedure was then repeated on the right eye but without reapplying the eye shield. B-scans were then collected over the next 11 minutes on the right eye without any hydration or mechanical blinking. Lens opacity onset occurred as early as 1.5 minutes (white arrows) and increased linearly over time (FIG. 3). After 11 minutes of observation and imaging, a drop of Systane Ultra was administered to the right eye and the protective eye shield reapplied. Ten minutes later, the eye shield and fluid were once again removed and another B-scan of the anterior segment was collected. A quantitative comparison between OCT images at 11 and 23 minutes reveals that the right eye opacity appears to slightly recede after being hydrated and covered. This is further supported by the quantitative data presented in FIG. 13, which illustrates stabilization and slight reduction (˜20%) of the in-depth scattering signal magnitude between 11 and 23 minutes.

Also shown in FIG. 12 is data collected from the left eye that remained protected for the entire duration of the time used to acquire all the images collected from the right eye. After being in place for 25 min., the eye shield on the left eye was removed and a B-scan of the anterior segment was collected. No apparent lens opacity developed during this period and in fact, opacity levels observed at the later time were below those observed at baseline.

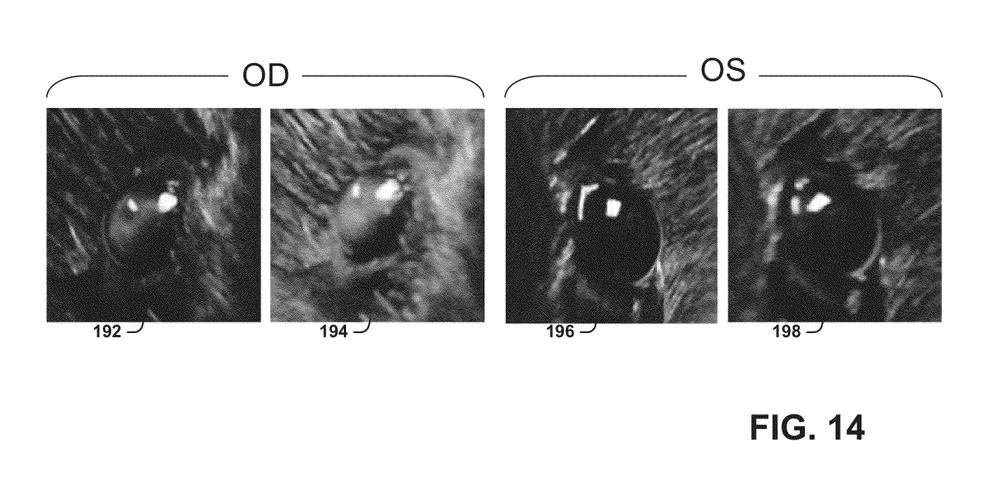

FIG. 13 contains a display of the scattering intensity changes related to opacity progression as a function of time. The in-depth signal amplitude from the anterior lens region of the left eye was on average ˜35% lower than that observed at baseline after 25 minutes of eye shield use but this difference was not statistically significant. FIG. 14 shows digital microphotograph examples of unprotected (with media opacity) and protected (without media opacity) mouse eyes collected after performing the experiment shown in FIGS. 12 and 13.

From the above description, those skilled in the art will perceive improvements, changes and modifications. Such improvements, changes and modifications are within the skill of one in the art and are intended to be covered by the appended claims.

What is claimed is:

1. A protective eye cup that protects an eye of a small animal from developing an ocular opacity in the refractive lens during a vision research experiment, the protective eye cup comprising:
a dome shaped with an outer geometry configured to facilitate placement and removal of the protective eye cup and a hemispherical inner geometry with an overall dome height and a radius of the dome opening of the dome selected to cover the eye,
wherein the inner geometry defines a hollow reservoir configured to hold the eye and an associated hydrating fluid when positioned on the eye; and
a shoulder configured to provide a radial edge to the dome that increases a contact surface area of the portion of the dome in contact with the hydrating fluid,
wherein the protective eye cup attaches to a region external to the eye based on a surface tension of the hydrating fluid.

2. The protective eye cup of claim 1, further comprising a post, that provides a grasping point for manipulation of the protective eye cup,
wherein the post is at least one of:
located directly over the top of the dome; and
located offset from the top of the dome on a side of the dome.

3. The protective eye cup of claim 1, wherein the hydrating fluid comprises at least one of: an ophthalmic fluid; one or more ophthalmic drops; and an ophthalmic ointment, and
wherein the hydrating fluid is aqueous or semi-viscous.

4. The protective eye cup of claim 1, wherein the hydrating fluid comprises at least one of Phosphate Buffered Saline (PBS), Balanced Salt Solutions (BSS), polyethylene glycol (PEG), propylene glycol, carboxymethylcellulose sodium, hypromethylcellulose, petroleum, mineral oil, Polymyxin B, or Bacitracin Zinc.

5. The protective eye cup of claim 1, wherein the outer geometry is at least one of: a hemispherical geometry, an ellipsoidal geometry, a hyperbolic geometry, a symmetrical polygonal geometry, a square geometry, and a rectangular geometry.

6. The protective eye cup of claim 1, wherein the small animal is a mouse, and
wherein the overall dome height is equal to the radius of the opening.

7. The protective eye cup of claim 1, wherein the small animal is a rat, and
wherein the overall dome height is less than the radius of the opening.

8. The protective eye cup of claim 1, wherein the shoulder comprises a round geometry, a square geometry, a rectangular geometry or a polygonal geometry, and
wherein the radial edge of the dome provided by the shoulder is substantially smooth.

9. The protective eye cup of claim 1, wherein the dome is at least one of opaque to light to shield the eye and optically transparent to permit imaging through the protective eye cup; and
wherein the hydrating fluid is at least one of pigmented black to shield the eye and optically transparent to facilitate imaging through the hydrating fluid.

10. A method for protecting an eye of a small animal from developing an ocular opacity in the refractive lens during a vision research experiment, the method comprising the steps of:
anesthetizing the small animal;
applying a hydrating fluid to the eye; and
covering the hydrating fluid and the eye with a protective eye cup,
wherein the protective eye cup comprises:
a dome shaped with an outer geometry configured to facilitate placement and removal of the protective eye cup from the eye and a hemispherical inner geometry with an overall dome height and a radius of the dome opening of the dome selected to cover the eye,
wherein the inner geometry defines a hollow reservoir configured to hold the eye and the hydrating fluid when positioned on the eye; and
a shoulder configured to provide a radial edge to the dome that increases a contact surface area of the portion of the dome in contact with the hydrating fluid,
wherein the protective eye cup attaches to a region external to the eye based on a surface tension of the hydrating fluid.

11. The method of claim 10, further comprising:
removing the protective eye cup from the eye;
refreshing the hydrating fluid covering the eye; and
imaging the eye.

12. The method of claim 11, further comprising refreshing the hydrating fluid when the time period for the imaging is 90 seconds or more.

13. The method of claim 11, further comprising placing the protective eye cup on the eye at the conclusion of the imaging.

14. The method of claim 13, further comprising:
removing the protective eye cup from the eye;
removing excess hydrating fluid from the eye;
applying an ointment to the eye; and
placing the protective eye cup on the eye after the application of the ointment.

15. The method of claim 14, further comprising discarding the protective eye cup when the small animal wakes from the anesthetic and knocks the protective eye cup from the eye.

16. A system that protects an eye of a small animal from developing an ocular opacity in the refractive lens during a vision research experiment, the system comprising:
a hydrating fluid that moistens the eye; and
a protective eye cup configured to hold the hydrating fluid and the eye when positioned on the eye,
wherein the protective eye cup attaches to a region external to the eye based on a surface tension of the hydrating fluid and covers the entire eye of the small animal.

17. The system of claim 16, wherein the protective eye cup comprises a dome shaped with an outer geometry configured to facilitate placement and removal of the protective eye cup on the eye and a hemispherical inner geometry with an overall dome height and a radius of the dome opening of the dome selected to cover the eye.

18. The system of claim 17, wherein the inner geometry defines a hollow reservoir configured to hold the eye and the hydrating fluid when the protective eye cup is positioned on the eye.

19. The system of claim 17, wherein the protective eye cup comprises a shoulder configured to provide a radial edge to the dome that increases a contact surface area of the portion of the dome in contact with the hydrating fluid.

20. The system of claim 16, wherein the protective eye cup comprises a post that provides a grasping point for manipulation of the protective eye cup.
